(19) United States
(12) Patent Application Publication
(10) Pub. No.: US 2020/0253659 A1
(43) Pub. Date: Aug. 13, 2020

(54) Diabetes Treatment Methods and Devices

(71) Applicant: Metaboscopy Medical, Inc., San Jose, CA (US)

(72) Inventors: Tsung-Chun Lee, San Jose, CA (US); Cheng-Tung Huang, San Jose, CA (US)

(73) Assignee: Metaboscopy Medical, Inc., San Jose, CA (US)

(21) Appl. No.: 16/861,749

(22) Filed: Apr. 29, 2020

Related U.S. Application Data
(63) Continuation of application No. PCT/US2018/057842, filed on Oct. 26, 2018.
(60) Provisional application No. 62/579,028, filed on Oct. 30, 2017.

(51) Int. Cl. A61B 18/04; A61M 25/10; A61B 1/00

(52) U.S. Cl. CPC A61B 18/04; A61M 25/1011; A61B 2018/00494; A61M 2210/1057; A61B 1/00082

(57) ABSTRACT

Endoluminal devices and methods that facilitate treatment of a desired treatment region of the gastrointestinal tract, in particular the duodenum, are provided herein. Such devices include a catheter having a treatment delivery portion disposed between proximal and distal balloons. The treatment can include thermal ablation of the treatment region by delivering a treatment fluid to the treatment region between the inflated balloons. In one aspect, the treatment fluid is delivered so as to fill the entire treatment region between the balloons without regard to the inflated balloon pressure. To ensure filling, the treatment fluid can be delivered into the treatment region until a pressure increase is observed or until a pre-determined volume is delivered. Other treatment devices and methods include means of uniformly distributing a treatment gas for plasma ablation, electrical ablation energy or a chemical or drug eluting stent.

### CROSS-REFERENCES TO RELATED APPLICATIONS

The present application is a Continuation of PCT/US2018/057842 filed Oct. 26, 2018; which claims the benefit of U.S. Provisional Appln No. 62/579,028 filed Oct. 30, 2017; the contents of which are incorporated herein by reference in their entirety for all purposes.

### BACKGROUND OF THE INVENTION

The small intestine is the body's largest hormone-producing organ, and hormones produced in the small intestine have long been known to play an important role in blood glucose regulation. Recent discoveries in metabolic science are now demonstrating that changes to the lining of the first segment of the small intestine—the duodenum—are associated with common metabolic disorders like type 2 diabetes mellitus, non-alcoholic steatohepatitis, as well as obesity.

After food passes through the stomach, it moves to the duodenum, which is the first part of the small intestine and the region where nutrient absorption begins in the gastrointestinal tract. The lining of the small intestine, or mucosa, is composed of several cell types, including hormone-producing cells (enteroendocrine cells). These hormone-producing cells (especially in the duodenum) sense the presence or absence of nutrients in the duodenum and send chemical signals to the body to help regulate insulin production and mediate glucose control.

Diets high in sugar and fat can cause significant changes in the duodenum over time, resulting in a thickened mucosa, abnormal nutrient absorption and alterations in the type and number of hormones released from the duodenum into the body, including hormones that help control insulin secretion and glucose homeostasis. This irregular chemical signaling is an important contributor to insulin resistance, which can develop into type 2 diabetes mellitus and other metabolic diseases such as non-alcoholic steatohepatitis.

Currently, there are varied treatment approaches to gastrointestinal related disorders. These approaches include devices that occupy space within the intestinal tract, such as intragastric balloons, transpyloric shuttles and devices that induce satiety; surgical interventions that include aspiration devices, gastric sleeves or pouches, gastrectomy, and Roux-en-Y bypass surgery; and implants, such as bypass sleeves. Each of these approaches have considerable drawbacks, including patient discomfort and nausea, erosion of the gastrointestinal tract, ulcers and bleeding, as well as implant migration and removal. In addition, conventional approaches have shown limited success with patient outcomes varying considerably across patient populations. There is a need for improved treatment devices and methods that can be effected in a minimally invasive procedure, that have minimal side effects, a shortened procedure and recovery time, and that are effective for longer durations of time, such as six months or more.

In recent years, small bowel interventional techniques have been developed that resurface the mucosal surface of the duodenum. Such techniques include ablation of the superficial duodenal mucosa after lifting of the surface by injection with a submucosal saline injection. While such techniques have shown promising patient outcomes, such procedures are painful, and require a series of time-consuming, complex steps, taking up to 40 minutes or more in performing such procedures. In addition, given the complexity of the procedure, inconsistencies in various steps can have adverse affects on patient outcomes. For example, if portions of the superficial duodenal mucosa are not lifted properly along the entire length of the treated portion of the duodenum, the treatment can result in incomplete resurfacing. Additionally, the surface of the lifted mucosa is highly irregular such that subsequent ablation still may not completely resurface the treated area, which can result in incomplete ablation resurfacing and repopulation of the treated area with errant mucosal cells. These drawbacks can result in inconsistent patient outcomes and require repeated procedures over time.

There is a need for improve devices and methods that allow for treatment of a portion of the gastroinstentinal tract in a minimally invasive manner with minimal pain and side effects for the patient and that can be effected by the clinician with greater use of use. There is further need for treatment methods that allow for shortened procedures, that can be performed more reliably and with greater consistency in patient outcomes, and that are effective long term.

### BRIEF SUMMARY OF THE INVENTION

This application generally relates generally to treatment devices and methods of treating disorders related to the gastrointestinal tract, in particular type 2 diabetes mellitus and non-alcoholic steatohepatitis.

In one aspect, the invention pertains to a method of treating a gastrointestinal disorder of a patient, such as type 2 diabetes mellitus. Such methods can include advancing a first catheter through a gastrointestinal tract of the patient to position a distal treatment region within a duodenum of the patient. In some embodiments, the distal treatment region includes a treatment delivery portion disposed between a proximal balloon and a distal balloon. The treatment delivery portion includes one or more delivery openings fluidly coupled with a delivery lumen extending to a proximal region of the first catheter. Next, the distal treatment region is positioned within a desired treatment region of the duodenum. The proximal and distal balloons are then inflated so as to sealingly engage an inside surface of the duodenum at opposite ends of the treatment region of the duodenum. Typically, the balloons are inflated by only a slight positive pressure. The treatment fluid is then delivered through the one or more openings while the proximal and distal balloons are sealingly engaged with the inside surface of the duodenum. Next, the treatment region of the duodenum between the inflated balloons is substantially filled with the treatment fluid. The treatment fluid is then maintained within the treatment region of the duodenum for a duration of time sufficient to treat the treatment region of the duodenum. It is appreciated that the treatment could be applied to other parts of the small intestine (e.g. jejunum) or other body lumens as well.

In some embodiments, the treatment fluid is maintained at a suitable temperature for thermally treating the treatment region of the duodenum. This can be accomplished by delivering the treatment fluid at a temperature elevated above the lowest suitable temperature or by monitoring the temperature of the treatment fluid and adjusting the temperature as needed.

The treatment fluid can be any suitable liquid (e.g. water, saline, alcohol, acetic acid or combinations) that is heated to a temperature within a range between 40 degrees Celsius and 100 degrees Celsius so as to thermally ablate a superficial lining of the treatment region of the duodenum. In some embodiments, the temperature range is between 50-100 degrees Celsius, 60-100 degrees Celsius, 70-100 degrees Celsius, or 70-95 degrees Celsius. Typically, the duration of time for such a heated fluid is at least thirty seconds. In some embodiments, the duration of time is at least one minute, two minutes, three minutes, or any duration up to 10 minutes or more. It is appreciated that, in some embodiments, the duration may vary based on the temperature of the treatment fluid, see for example the contours in FIGS. 16A-16B. It is appreciated that, in some embodiments, the duration may be largely independent from variations in temperature so long as the temperature exceeds body temperature. In some embodiments, the proximal and distal balloons are filled with air or fluid to insulate the treatment fluid and maintain the suitable temperature for the thermal treatment. In some embodiments, the balloons are filled with air, which insulates the heated fluid so as to allow a target temperature of the treatment fluid to be more easily maintained during treatment. In some embodiments, the proximal and distal balloons have a diameter between 1.5 cm and 4 cm and can be formed of a semi-compliant material so as to sealingly engage with an inner wall of the duodenum.

In one aspect, the treatment fluid is delivered into the treatment region of the duodenum between the inflated balloons so as to substantially fill the treatment fluid without regard to a pressure within the proximal and distal balloons. In some embodiments, filling of the treatment region between the inflated balloons is performed by delivering the treatment fluid until a pre-determined pressure increment (e.g. 0.5 atm) from a baseline of the delivery pressure or pressure within the duodenum is observed. In some embodiments, the treatment fluid is delivered until a pre-determined volume of fluid is delivered.

In another aspect, endoluminal devices that facilitate treating a gastrointestinal disorder of a patient with a treatment fluid provided herein. Such devices can include a first catheter extending from a proximal end to a distal treatment delivery region thereof. The distal treatment delivery region can include a treatment delivery portion disposed between a proximal balloon and a distal balloon. The treatment delivery portion can include one or more delivery openings in fluid communication with a delivery lumen extending to the proximal end of the first catheter. The proximal and distal balloons are fluidly coupled with one or more inflation lumens extending to the proximal end of the first catheter. Typically, the proximal and distal balloons are spaced apart by a fixed distance suitable for treatment of a desired treatment region of a duodenum of the patient. In some embodiments, the fixed distance is between 5 and 15 cm in length so as to allow concurrent treatment of a portion of the duodenum suitable for treatment of type 2 diabetes mellitus or non-alcoholic steatohepatitis. In some embodiments, each of the proximal and distal balloons is between 1.5 and 4 cm in diameter to facilitate sealing engagement with the inside surface of the duodenum. Typically, the proximal and distal balloons are formed of a semi-compliant material. In some embodiments, the device can include a temperature sensor disposed along the treatment delivery portion. Such devices can further include a heater configured for heating the treatment fluid and a controller configured to adjust heating of the treatment fluid with the heater based on an output from the temperature according to a control loop. In some embodiments, the device further includes one or more aspiration ports to aspirate or circulate the treatment fluid within the treatment region or a circulator configured for circulating the treatment fluid.

In yet another aspect, endoluminal devices that facilitate treatment of a gastrointestinal disorder of a patient with various other means are provided herein. Such various other means include plasma ablation, electrical ablation, chemical ablation and treatment by a therapeutic from an implant, such as a stent. Some such devices include expandable structures or balloons that facilitate uniform release of a treatment gas, such as argon, to generate an ablating plasma along the entire treatment region. Other such devices include electrode balloons to ablate the superficial mucosa with electrical ablating energy. Still, other such device can include expandable structures, such as a stent, that gradually release a chemical or therapeutic to chemically ablate or treat the treatment region of the duodenum. In some embodiments, the above expandable structures are configured to treat the entire treatment region without requiring movement of the device during treatment delivery. Methods of delivering treatment utilizing the devices described herein are also provided.

### BRIEF DESCRIPTION OF THE DRAWINGS

FIG. 1 shows an overview of an endoluminal treatment device during delivery of treatment, in accordance with some embodiments of the invention;

FIG. 2 illustrates the anatomy of the gastrointestinal treatment;

FIGS. 3A and 3B show an example treatment device in accordance with some embodiments;

FIGS. 4-8 show example treatment devices in accordance with some embodiments;

FIGS. 9-11 show example argon ablation treatment devices in accordance with some embodiments;

FIG. 12 shows an example argon ablation treatment device having an expandable basket in accordance with some embodiments;

FIGS. 13A-13C show examples of an expandable basket during delivery, deployment and treatment, in accordance with some embodiments;

FIG. 14 shows an example RF ablation treatment device having an expandable electrode balloon in accordance with some embodiments;

FIG. 15 shows an example treatment device having an expandable drug eluting stent in accordance with some embodiments; and

FIGS. 16A-16B show animal study data demonstrating the relationship between tissue injury, treatment fluid temperature and duration of exposure.

### DETAILED DESCRIPTION OF THE INVENTION

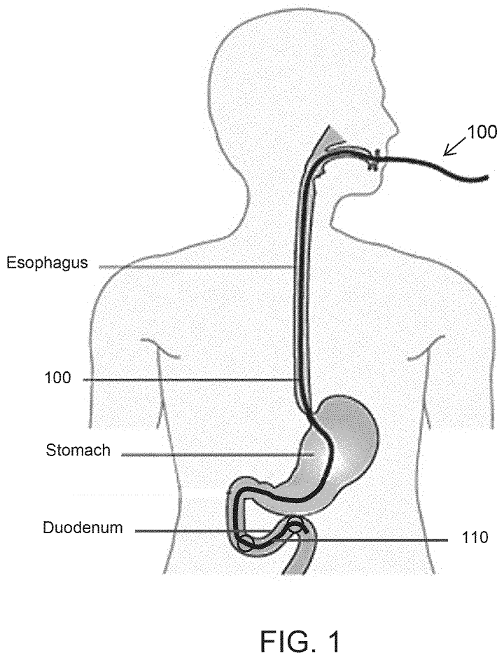

The invention pertains to non-invasive endoluminal devices that are advanced along the gastrointestinal tract via natural orifices. Typically, as shown in FIG. 1, the endoluminal device 100 is inserted into the patient orally and advanced through the patient's esophagus and stomach and into the duodenum to position a distal treatment region 110 within a desired treatment region 10 of the duodenum. The device delivers treatment to a superficial mucosal surface of a portion of the duodenum. In some embodiments, the device deliver therapeutic energy to so as to alter or ablate a superficial mucosal surface of the duodenum along a desired treatment region, typically a continuous surface within a region of the duodenum extending within the second or third portions of the duodenum, which are advantageously downstream of the Ampulla of Vater so as to avoid potential blockage of the openings of bile duct or pancreatic duct.

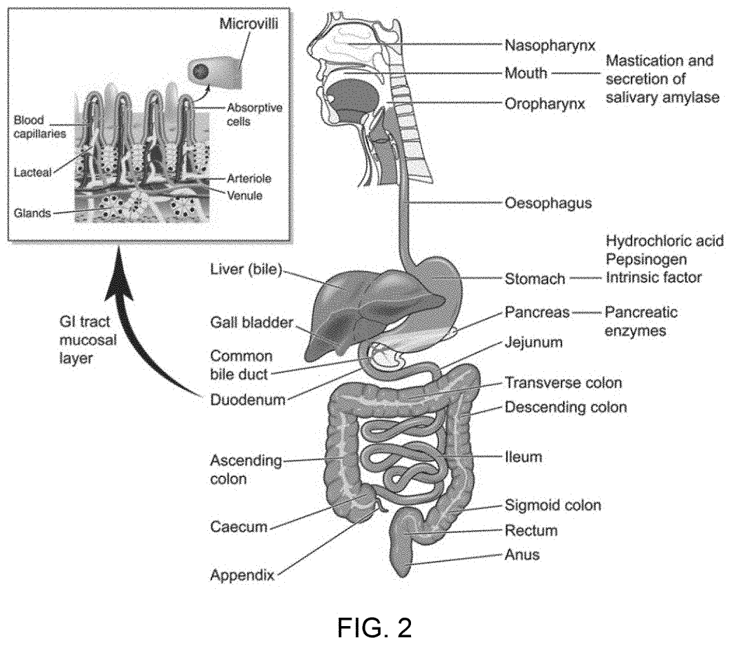

As can be seen in FIG. 2, the superficial mucosal surface of the duodenum contains various enteroendocrine cells, particularly L-cells, that release hormones when exposed to digested matter passing through the gastrointestinal tract. An imbalanced hormonal reaction by L-cells to ingesting food leads to insulin resistance and subsequent poor regulation of blood sugar (type 2 diabetes mellitus) and non-alcoholic steatohepatitis. Ablation of these cells within the superficial mucosal layer along a portion of the small intestine (e.g. the duodenum), typically about 8 to 10 cm in length, can stimulate the regeneration of the small intestinal mucosa and restore a more regular enteroendocrine cell population along the small intestine, thereby improving blood glucose regulation (e.g. insulin resistance), reversing type 2 diabetes mellitus, as well as providing therapeutic effects on other diseases related to insulin resistance (e.g. non-alcoholic steatohepatitis (NASH, so called Fatty Liver). It is understood that ablation, as referred to herein, can refer to thermal ablation (e.g. heating or cooling), plasma ablation, or chemical ablation. Treatment can also include introduction of a therapeutic agent (e.g. absolute alcohol, acetic acid or combination). Typically, the treatment region is within a second or third portion of the duodenum below the Ampulla of Vater, (the outflow port of common bile duct/pancreatic duct to the duodenum), so as to avoid complications of infection due to blockage of the bile ducts or the pancreatic duct.

In one aspect, the invention pertains to an endoluminal catheter device having a distal region having a treatment delivery portion disposed between proximal and distal balloons. The overall length of the device from a proximal hub to the distal tip is sufficiently long, for example about 130 cm, so that the distal treatment delivery portion can be positioned within the desired treatment region of the duodenum while the hub remains readily accessible by the clinician. The treatment delivery portion can include one or more openings in fluid communication with a delivery lumen to facilitate delivery of the treatment fluid into a region of the duodenum between inflated proximal and distal balloons sealingly engaged within the duodenum. The balloons are of a size, shape and material suitable for inflation within the duodenum and to facilitate sealing engagement within the inner wall of the duodenum. The balloons can be formed of a compliant, non-compliant, or semi-compliant material or combinations thereof. Typically, the balloons are formed of a semi-compliant material so as to assume a rounded shape while still providing some conformance with the inner wall of the duodenum, thereby allowing for improving sealing within the duodenum. In some embodiments, each of the balloons have a diameter within a range of 1.5 cm and 4 cm, such as between 2 cm and 4 cm, so as to substantially fill and seal within the duodenum. In some embodiments, the balloons are formed of a clear or translucent material so as to allow the treatment region to be viewed through the balloons with an endoscope. In some embodiments, the balloons include a radiopaque marker to allow the bounds of the treatment region defined by the balloons to be readily determined through standard X-ray visualization techniques. The treatment deliver portion can include multiple openings for delivery and/or aspiration of the treatment fluid, and can further include one or more sensors for detecting a temperature of the treatment fluid. In some embodiments, the treatment delivery portion includes a heating or cooling device for actively controlling a temperature of the treatment fluid and can further include a circulator device to circulate the treatment fluid within the treatment region to facilitate a more uniform temperature distribution during treatment.

In some such devices, each of the proximal and distal balloons is inflatable so as to sealingly engage the walls of the duodenum, which are typically 20-30 mm in diameter. The balloons are spaced apart by a fixed length to define a treatment area between the inflated balloons, typically a portion of the duodenum about 8 to 10 cm in length. In some embodiments, the balloons are inflated with only a slight positive pressure (e.g. 1 psi-14 psi above ambient pressure). In some embodiments, the balloons are positionable such that the desired treatment area can be adjusted to a desired length. Each of the proximal and distal balloons can be fed by a separate lumen that extends to a proximal end of the device so as to be independently inflatable, or can be fed from a common inflation lumen so as to facilitate concurrent inflation and equalize pressure.

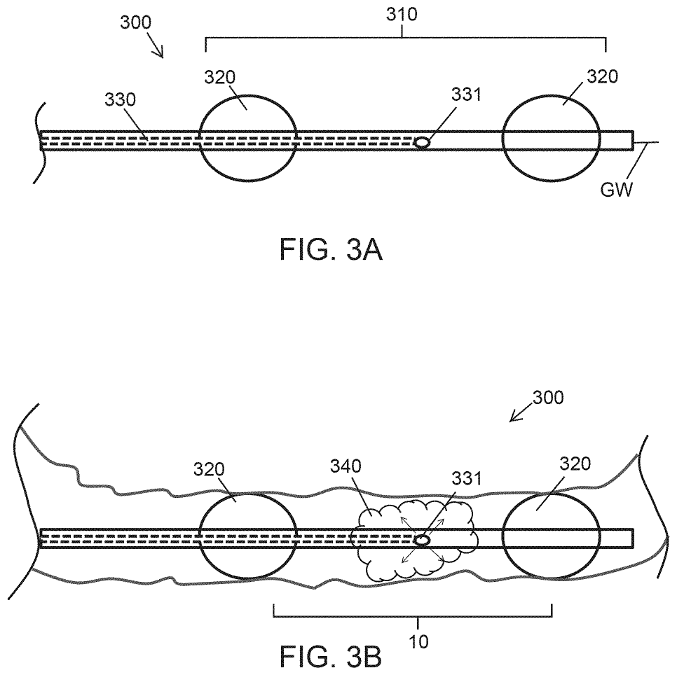

In such devices, the treatment delivery portion can include one or more outlets for delivery of an ablation agent, such as a hot fluid (e.g. water or vapor), into the portion of the duodenum sealed between the proximal and distal balloons. The outlets are fed by a lumen that extends to the proximal end of the device into which a clinician delivers the heated treatment fluid. The temperature of the fluid can be determined/monitored externally, or can be monitored by a sensor disposed on a distal portion of the device between the proximal and distal balloons. In some embodiments, the treated area can include a thermocouple in the treatment area to monitor temperature and a circulation loop to control water temperature by two or multiple inflow and outlet channels. The device can also include a localized heating element (e.g. a localized heating apparatus at the distal tip of fluid outflow channel) within the treatment area to generate or maintain the heated water or vapor within the treatment area for the duration of the treatment. The heated treatment fluid can be delivered so as to substantially fill the entire treatment region of the duodenum between the proximal and distal inflated balloons. The heated fluid is maintained for a period of time sufficient to ablate the superficial duodenal mucosa along the treatment area, and stimulate the regeneration of normalized population of enteroendocrine cells (e.g. L-cells) that are related to poor blood sugar regulation (type 2 diabetes mellitus) and other metabolic diseases (non-alcoholic steatohepatitis). The outlets can also be used as inlets to aspirate the heated fluid/vapor after treatment. In some embodiments, the device can include a nosecone for release of contrast media to determine whether the balloons are sufficiently sealed against the vessel walls of the duodenum. Example embodiments of such devices are shown in FIGS. 3A-3B, and FIGS. 4-8.

FIGS. 3A and 3B show endoluminal catheter device 300 having a distal treatment region 310 that includes proximal and distal balloons 320 between which is disposed a treatment delivery opening 331 in fluid communication with a treatment fluid delivery lumen 330. In this embodiment, balloons 320 are axially separate by a fixed distance, typically between 5 cm and 15 cm (center-to-center distance), typically about 8-10 cm such that a corresponding length of the duodenum is treated by delivery of the treatment fluid 340 the treatment fluid maintained in the treatment region 10 defined between the two balloons when inflated within the duodenum, as shown in FIG. 3B. Catheter device can further include a guidewire lumen extending therethrough to allow advancement of the device over a guidewire GW to facilitate delivery of the device and positioning of the treatment delivery portion 310 within the treatment region.

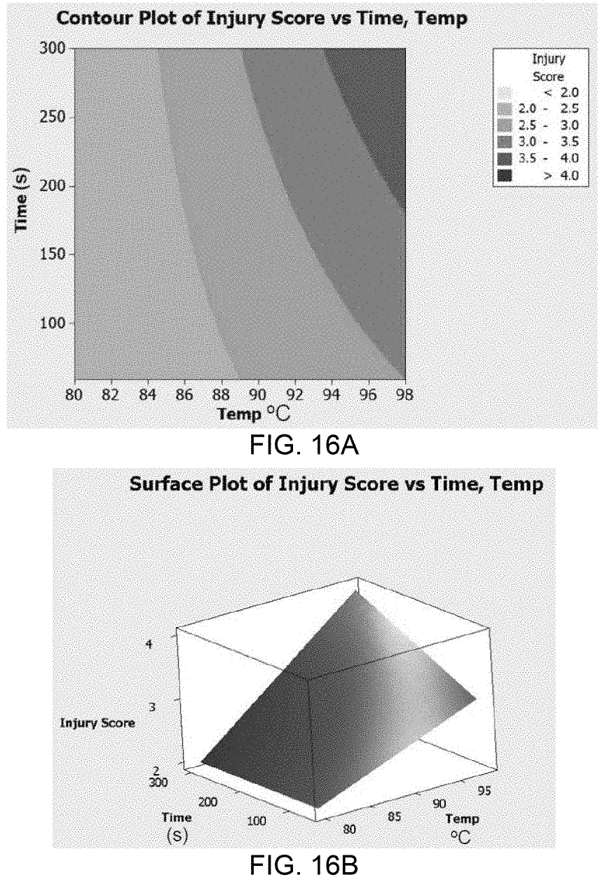

In some embodiments, the treatment fluid 340 is a fluid (e.g. water, saline) that is heated to a temperature sufficient to thermally ablate the superficial mucosa. Typically, the treatment fluid is a temperature within a range between 40 degrees and 100 degrees Celsius to facilitate thermal ablation of the superficial mucosa while avoiding unnecessary damage to underlying tissues. In some embodiments, the temperature range can be between 60 degrees and 100 degrees Celsius. Preliminary animal studies indicate a particularly therapeutic effect within a temperature range from 70 degrees and 100 degrees Celsius, preferably between 80 degrees and 95 degrees Celsius. Preferably, the superficial mucosa is maintained above 60 degrees Celsius for at least thirty seconds to ensure ablation of the superficial mucosa. In some embodiments, the duration can be one minute, two minutes, three minutes or any duration up to 10 minutes or more to ensure sufficient cell exposure. It is desirable to avoid ablating deeper tissues underlying the superficial mucosa as this can result in damages to nearby organs, such as the pancreas causing pancreatitis or damages to deeper intestinal tissues resulting in scarring and formation of stenosis with subsequent narrowing of the duodenum and stenosis complication. Since the temperature of the heated fluid tends to drop upon initial introduction into the treatment region, it is desirable for the treatment fluid to be maintained at a temperature above 60 degrees Celsius, for example between 80 degrees and 100 degrees Celsius. The heated treatment fluid is maintained for a period of time greater than 30 seconds, for example one or more minutes, typically about three minutes or more so as to ensure the expose superficial mucosa is sufficiently heated to ablate the errant cells. In some embodiments, the treatment can include a treatment fluid at a temperature within a range from 80 degrees to 95 degrees Celsius at a duration of at least 30 seconds or more, which thus far has demonstrated a robust and consistent therapeutic response, as demonstrated by FIGS. 16A-16B. While heated fluid is described here, it is appreciated that in other embodiments, the treatment fluid can be a cooling liquid (e.g. cryotherapy), a chemical to chemically ablate the mucosa, and can include a therapeutic to treat the errant cells or facilitate healing and repopulation of the superficial mucosa after treatment. It is further appreciated that the treatment can include a combination of any of these aspects (e.g. cooling after heating, thermal treatment fluid having a chemical or therapeutic compound).

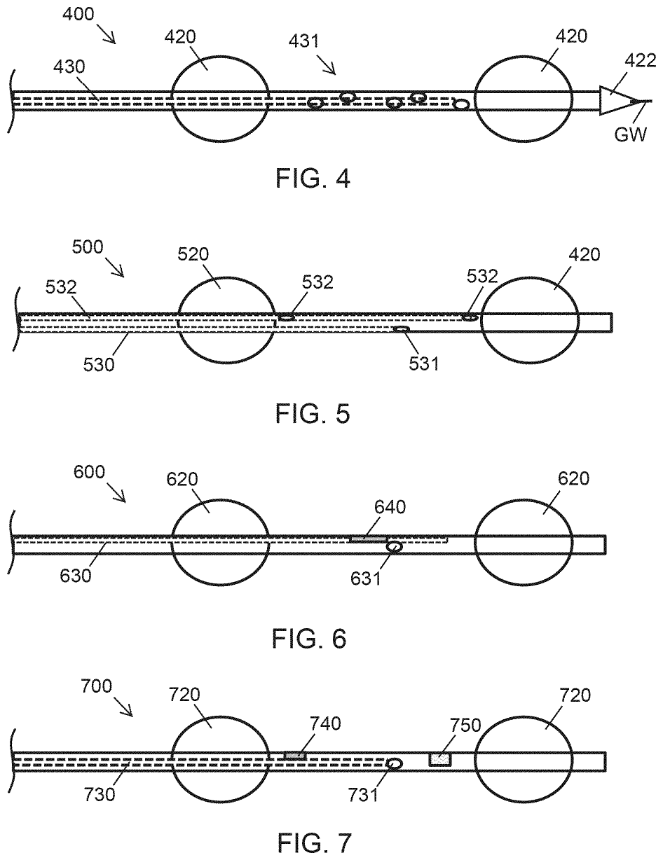

FIG. 4 shows endoluminal catheter device 400, which includes proximal and distal balloons 420 and multiple openings 431 in fluid communication with treatment delivery lumen 430. Openings 431 can distributed along the region between balloons 420 and can further be arranged in a pattern (e.g. helical) to facilitate circulation of the fluid during delivery. In this embodiment, a distal nosecone 422 is included, which facilitates introduction of the device along guidewire GW. In some embodiments, the guidewire lumen can be used as the treatment delivery lumen by utilizing a nosecone 422 that blocks the distal exit of the guidewire lumen.

FIG. 5 shows endoluminal catheter device 500, which includes proximal and distal balloons 520 and a treatment delivery opening 531 in fluid communication with a delivery lumen 530. The device further includes an aspiration opening 532 in fluid communication with an aspiration lumen 532, which allows for subsequent aspiration after treatment or can allow for circulation by aspirating the treatment fluid concurrent with delivery so as to provide a more uniform temperature profile during treatment. The depicted device includes a pair of aspiration openings 532 spaced apart from the delivery opening and disposed at each end to facilitate complete aspiration after treatment or to provide a circulation path within the treatment region.

FIG. 6 shows endoluminal catheter device 600, which includes proximal and distal balloons 620 and a treatment delivery opening 631 in fluid communication with a delivery lumen 630. The device further includes a temperature sensor 640 (e.g. thermocouple) disposed within the treatment region defined between the balloons 620. The sensor can be used to ensure the temperature within the treatment region is maintained within the target temperature range for a specified duration of time. The sensor can also be communicatively coupled with a treatment fluid temperature control, for example within an external fluid reservoir, to allow adjustment of the treatment fluid being delivered into the treatment region such as in a control loop.

FIG. 7 shows endoluminal catheter device 700, which includes proximal and distal balloons 720 and a treatment delivery opening 731 in fluid communication with a delivery lumen 730. The device further includes a temperature sensor 740 (e.g. thermocouple) and a fluid heater 750 disposed within the treatment region defined between the balloons 620. The fluid heater 750 can be communicatively coupled to the sensor through a controller so that the treatment fluid within the region can be actively heated based on an output of the temperature sensor 740. In other embodiments, the device can include a fluid cooler configured to actively cool the treatment fluid either during or after treatment.

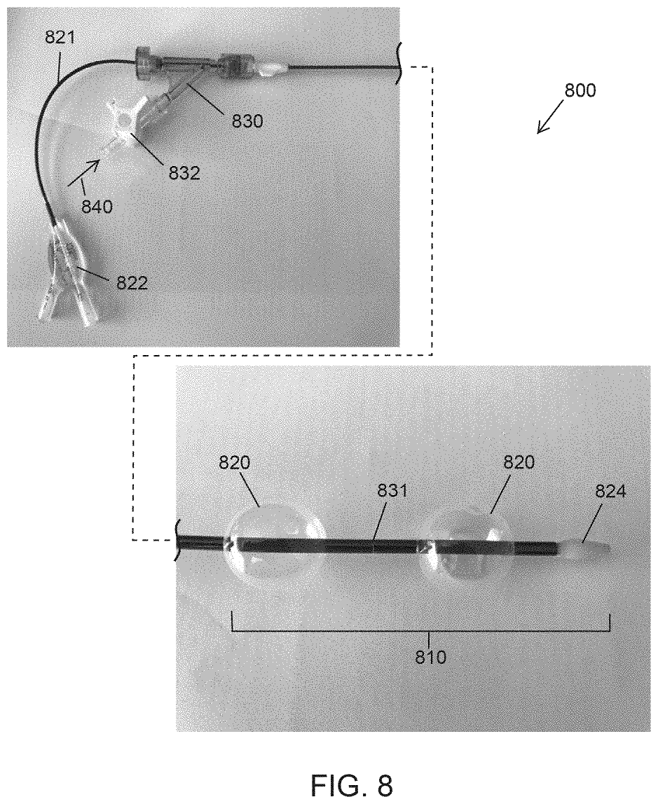

FIG. 8 shows an overview of an endoluminal catheter device 800 having a distal treatment delivery region 810 that includes a treatment delivery opening 831 between proximal and distal balloons 820 and a distal nosecone 824. The proximal end of the catheter device 800 includes a port/valve assembly to facilitate control of balloon inflation and introduction of the treatment fluid 840. This proximal assembly includes a pair of balloon inflation ports 822 that allow for independent balloon inflation, and an ablation agent port 832, which includes a manually controlled valve that allows a clinician to readily control introduction and termination of the treatment fluid.

In some embodiments, the ports can be coupled with manually controlled pump or syringe to allow manual control of balloon inflation and introduction of treatment fluid. In some embodiments, the clinician introduces a pre-determine volume of the treatment fluid, which can be estimated by the morphology and length of the treated region of the duodenum or can be determined by pre-filling the treatment region before treatment.

In other embodiments, a pressure monitor can be used, for example, an external pressure sensor or gage fluidly coupled with the treatment fluid delivery path. A pressure sensor can be disposed within the treatment region or can be fluidly coupled with the flowpath or reservoir and disposed outside the patient's body. By monitoring the pressure during delivery of the treatment fluid, the clinician can determine when the treatment region is substantially filled with the treatment fluid, typically without any regard to an inflation pressure within the balloons. For example, when the treatment fluid entirely fills the treated region of the duodenum, there is an increase in pressure in the delivery pressure or the pressure within the duodenum since the filled space is confined by the proximal and distal balloons. It is desirable to observe this increase in pressure before it becomes substantial so as to prevent leakage of the treatment fluid beyond the inflated balloons. Thus, in some embodiments, delivery of the treatment fluid is terminated when the pressure increases by a relatively small margin of the average delivery pressure, for example about 25% or less, such as about 10% or less, or about 5%. In some embodiments, the delivery pressure and/or the pressure within the duodenum is monitored in conjunction with monitoring a volume of treatment fluid delivered to determine an approaching end point as the treatment region becomes filled.

In another aspect, the treatment device can include various other means of ablating or treating the superficial mucosa, including the use of plasma ablation (e.g. argon), electrical ablation (e.g. RF), chemical ablation, or therapeutic treatment (e.g. drug eluting implants).

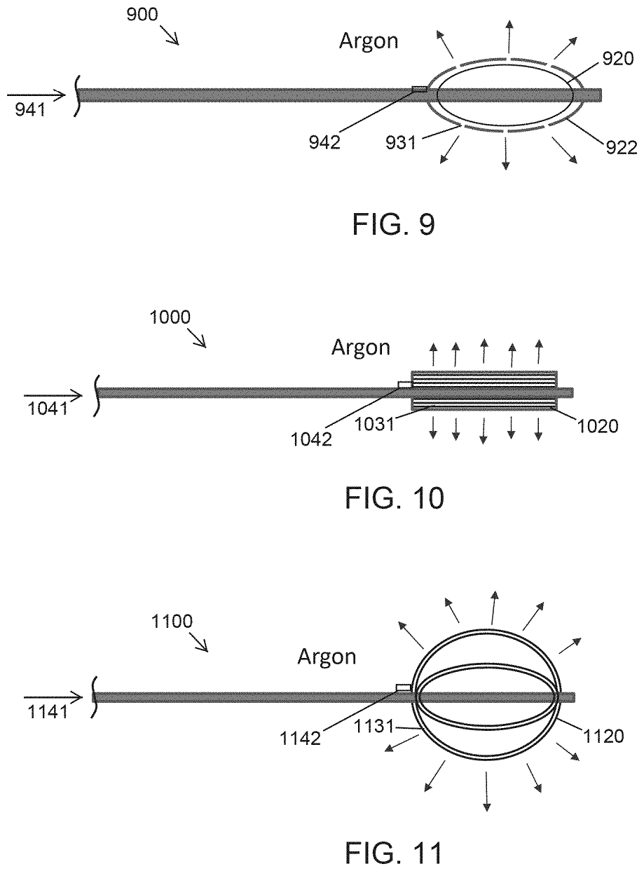

FIGS. 9-12 depict endoluminal catheter devices that utilizes plasma ablation to ablate the superficial mucosa within the duodenum. FIG. 9 depicts catheter device 900, which includes a distal treatment region having an inner balloon 920 and an outer balloon 920 with small release openings 931 defined therein to allow release of a suitable treatment gas (e.g. argon) introduced into a space between the inner and outer balloons. The inner balloon 920 is inflated so as to move the multiple openings 931 near the superficial mucosa to facilitate uniform and concurrent plasma ablation of the mucosa. One or more electrodes 942 can be used to activate the plasma after release of the gas. The balloons can be defined with suitable diameters for deployment within the duodenum, as described herein. In some embodiments, the inflated balloons are proximally retracted during delivery of plasma to facilitate ablation along the desired treatment region. In other embodiments, the balloons are of a sufficient length (e.g. 5-15 cm, 8-10 cm) to allow plasma ablation of the superficial mucosa in the treatment region without moving or repositioning the device during treatment delivery.

FIG. 10 depicts catheter device 1000, which includes a distal treatment region having a slotted sleeve or mesh 1020 having lengthwise slots 1031 to allow more even distribution of the treatment gas over a wider area and one or more electrodes 1042 to produce plasma to ablate the superficial mucosa. The sleeve or mesh can be outwardly expanded when positioned within the treatment region. In some embodiments, the tube or mesh is formed of Nitinol. The sleeve can be defined to assume a suitable diameter for deployment within the duodenum. In some embodiments, the distal treatment region is proximally retracted during delivery of treatment to facilitate ablation along the desired treatment region. In other embodiments, the sleeve 1020 is of sufficient length (e.g. 5-15 cm, 8-10 cm) to allow plasma ablation of the superficial mucosa in the treatment region without moving or repositioning the device during treatment delivery.

FIG. 11 depicts catheter device 1100, which includes a distal treatment region with an expandable basket structure 1120 formed of a tube or mesh having openings 1131 throughout to facilitate uniform release of the treatment gas and one or more electrodes 1142 to produce plasma to ablate the superficial mucosa. The basket structure 1120 expands so as to contact the superficial mucosa. In some embodiments, the basket structure 1120 is formed of a Nitinol tube or mesh. In some embodiments, the distal treatment region is proximally retracted during delivery of treatment to facilitate ablation along the desired treatment region. In other embodiments, the basket is of sufficient length (e.g. 5-15 cm, 8-10 cm) to allow plasma ablation of the superficial mucosa in the treatment region without moving or repositioning the device during treatment delivery.

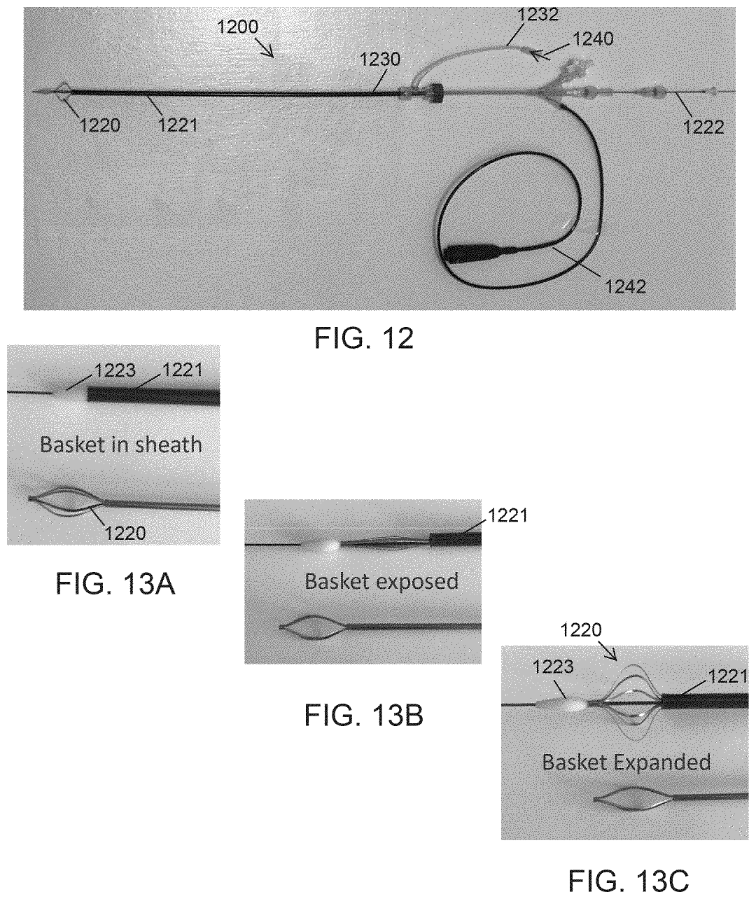

FIG. 12 depicts catheter device 1200, which includes an expandable basket 1220 that is constrained by a retractable sheath 1221. The sheath interfaces with a distal nosecone 1223 to facilitate delivery of the catheter into the target treatment region, as shown in FIG. 13A, and proximally retracts, as shown in FIG. 13B, after which the basket can be outwardly expanded by proximal retraction of the nosecone 1223, as shown in FIG. 13C. Once expanded, a suitable treatment gas 1240 can be introduced into port 1232 and through treatment delivery lumen 1230 for release through opening in tubing of the expandable basket 1220. The proximal retraction of the nosecone is facilitated by proximal shaft end 1222. Plasma generator connector 1242 electrically couples a distal electrode to plasma generator to produce the ablation plasma after introduction of the treatment gas during treatment. In some embodiments, the distal treatment region is proximally retracted during delivery of treatment to facilitate ablation along the desired treatment region. In other embodiments, the basket is of sufficient length (e.g. 5-15 cm, 8-10 cm) to allow plasma ablation of the superficial mucosa in the treatment region without moving or repositioning the device during treatment delivery.

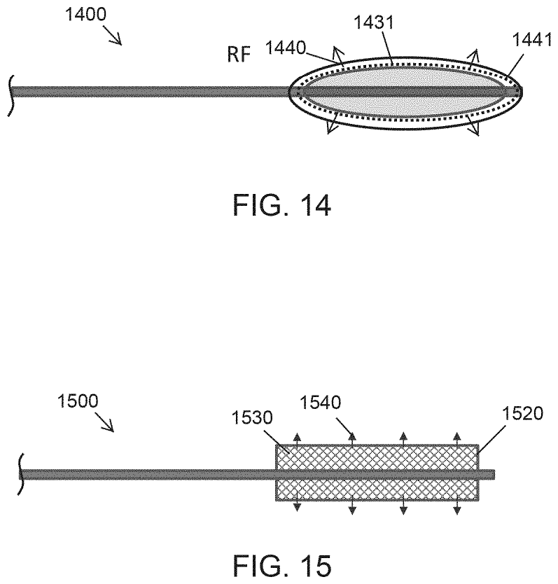

FIG. 14 depicts catheter device 1400, which includes a distal treatment region having an inflatable balloon 1431 with multiple ablating electrodes 1441 disposed thereon. After inflation of the balloon 1431 to contact the superficial mucosa within the duodenum, the electrodes are activated so as to deliver electrical ablating energy 1440 (e.g. RF energy) to the superficial mucosa. In some embodiments, the distal treatment region is proximally retracted during delivery of treatment to facilitate ablation along the desired treatment region. In other embodiments, the basket is of sufficient length (e.g. 5-15 cm, 8-10 cm) to allow plasma ablation of the superficial mucosa in the treatment region without moving or repositioning the device during treatment delivery.

FIG. 15 depicts catheter device 1500, which includes a distal treatment region configured to release a scaffold or stent 1520 that includes a coating 1530 of a treatment agent. The scaffold can be self-expanding or balloon expandable. The treatment agent can include a chemical and/or a therapeutic agent so as to chemically ablate or treat the superficial mucosa upon release of the treatment agent 1540. The stent 1520 is configured to assume a diameter suitable for deployment within the duodenum and is of a sufficient length (e.g. 5-15 cm, 8-10 cm) to treat the entire treatment region.

FIGS. 16A-16B show animal study data demonstrating therapeutic effect of the treatment approach of FIGS. 3A-3B. FIG. 16A shows a contour plot of tissue injury score versus time and temperature. Injury score refers to the severity grading scheme: 0=within normal limits, 1=minimal, 2=mild, 3=moderate, 4=severe. (Mann et al., 2012) and the Villi injury grade: 0=No damage, I=Occasion tips affected, II=Majority of tips affected, III=Majority of tips and some villi affected, IV=Tips, mid and lower portion of majority of villi affected. Effecting sufficient injury to cells of the superficial mucosa while avoiding injury to underlying tissues, ensures the regeneration of normalized population of enteroendocrine cells (e.g. L-cells) that are related to poor blood sugar regulation (type 2 diabetes mellitus) and other metabolic diseases (non-alcoholic steatohepatitis). The duodenum epithelium cells of the superficial mucosa typically regenerate within 2 to 5 days after the procedure, after which repopulation of the superficial mucosa occurs with L-cells regulated to normal levels. As demonstrated by the contour plot of FIG. 16A, a suitable injury score is attained within the range of about 85 degrees to 95 degrees Celsius at exposures of relatively short exposure durations such that about 30 seconds of exposure may be sufficient. However, to ensure that the cells of substantially the entire superficial mucosa of the treated region reach suitable temperatures, it may be desirable to prolong exposure to the treatment fluid to slightly longer periods of time, such as 2-3 minutes or more. FIG. 16B shows a surface plot of injury score versus time and temperature further demonstrating the relationship between tissue injury, exposure time and treatment fluid temperature.

While the exemplary embodiments have been described in some detail, by way of example and for clarity of understanding, those of skill in the art will recognize that a variety of modifications, adaptations, and changes may be employed. Hence, the scope of the present invention should be limited solely by the appending claims.

In the foregoing specification, the invention is described with reference to specific embodiments thereof, but those skilled in the art will recognize that the invention is not limited thereto. Various features, embodiments and aspects of the above-described invention can be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

1. A method of treating a gastrointestinal disorder of a patient, the method comprising:
advancing a first catheter through a gastrointestinal tract of the patient into a duodenum of the gastrointestinal tract, the first catheter having a distal treatment region with a treatment delivery portion disposed between a proximal balloon and a distal balloon, wherein the treatment delivery portion comprises one or more delivery openings fluidly coupled with a delivery lumen extending to a proximal region of the first catheter;
positioning the distal treatment region within a desired treatment region of the duodenum;
inflating each of the proximal and distal balloons, sealingly engage an inside surface of the duodenum at opposite ends of the treatment region of the duodenum;
delivering a treatment fluid through the one or more openings while the proximal and distal balloons are sealingly engaged with the inside surface of the duodenum;
substantially filling the treatment region of the duodenum between the proximal and distal balloons with the treatment fluid without regard to a pressure within the proximal and distal balloons; and
maintaining the treatment fluid within the treatment region of the duodenum for a duration of time sufficient to treat the treatment region of the duodenum.

2.-3. (canceled)

4. The method of claim 1, wherein each of the proximal and distal balloons are filled with air or a liquid suitable to insulate the treatment fluid and maintain the suitable temperature for the thermal treatment.

5. The method of claim 1, wherein the treatment fluid is a heated liquid between 60 degrees Celsius and 100 degrees Celsius so as to thermally ablate a superficial mucosal of the treatment region of the duodenum, and wherein the duration of time is at least thirty seconds.

6. The method of claim 5, wherein the thermal treatment thermally ablates the superficial mucosa of the treatment region of the duodenum in less than 10 minutes.

7.-8. (canceled)

9. The method of claim 1, wherein each of the proximal and distal balloons are formed of a semi-compliant material.

10. The method of claim 1, further comprising:
monitoring a treatment fluid delivery pressure during delivery of the treatment fluid with a pressure sensor or gauge.

11. The method of claim 10, wherein substantially filling the portion of the duodenum with the treatment fluid comprises delivering the treatment fluid until a pre-determined pressure increment from a baseline of a delivery pressure or a pressure within the duodenum is observed.

12. The method of claim 11, wherein the pressure increment from the baseline delivery pressure comprises about 0.5 atm, or between 0.5 atm to 1 atm, or between 1 atm to 2 atm, or between 2 atm and 5 atm.

13. The method of claim 1, wherein substantially filling the treatment region of the duodenum with the treatment fluid comprises delivering a pre-determined volume.

14. The method of claim 13, further comprising:
determining the pre-determined volume by pre-filling the treatment region of the duodenum between sealingly engaged, inflated proximal and distal balloons with a non-treatment fluid and recording the pre-determined volume.

15.-16. (canceled)

17. The method of claim 1, further comprising:
verifying sealing of the proximal and distal balloons by visualization techniques.

18. (canceled)

19. An endoluminal device for treating a gastrointestinal disorder of a patient, the device comprising:
a first catheter extending from a proximal end to a distal treatment delivery region thereof, wherein the distal treatment delivery region includes a treatment delivery portion disposed between a proximal balloon and a distal balloon,
wherein the treatment delivery portion comprises one or more delivery openings in fluid communication with a delivery lumen extending to the proximal end of the first catheter,
wherein the proximal and distal balloons are fluidly coupled with one or more inflation lumens extending to the proximal end of the first catheter,
wherein the proximal and distal balloons are spaced apart by a fixed distance suitable for treatment of a desired treatment region of a duodenum of the patient; and
a controller configured to control pressurization of the proximal and distal balloons and to control delivery of a heated treatment fluid through the one or more delivery opening to substantially fill treatment region of the patient's gastrointestinal tract between the proximal and distal balloons with the treatment fluid without regard to a pressure within the proximal and distal balloons to facilitate thermal ablation of superficial mucosa of the treatment region.

20. The endoluminal device of claim 19, wherein the fixed distance is between 5 and 15 cm in length.

21. (canceled)

22. The endoluminal device of claim 19, wherein each of the proximal and distal balloons are formed of a semi-compliant material.

23. (canceled)

24. The endoluminal device of claim 19, wherein the one or more delivery opening comprise a plurality of openings that are arranged to facilitate distribution or circulation of the treatment fluid during treatment.

25. The endoluminal device of claim 19, further comprising:
one or more aspiration openings in fluid communication with one or more aspiration lumens to facilitate circulation or aspiration of the treatment fluid.

26. The endoluminal device of claim 19, further comprising:
a temperature sensor disposed between the proximal and distal balloons;
a heater configured for heating the treatment fluid; and
wherein the controller is configured to adjust heating of the treatment fluid with the heater based on an output from the temperature according to a control loop.

27. The endoluminal device of claim 19, further comprising:
a circulator configured for circulating the treatment fluid within the treatment region between the proximal and distal balloons during treatment.

28. The endoluminal device of claim 19, wherein one or both of the proximal and distal balloons are clear or translucent to allow monitoring therethrough with an endoscope.

29.-41. (canceled)

42. The endoluminal device of claim 19, wherein the controller is configured to substantially fill the treatment region between the proximal and distal balloons by monitoring a treatment fluid delivery pressure during delivery of the treatment fluid with a pressure sensor or gauge.
